(19) United States
(12) Patent Application Publication
(10) Pub. No.: US 2019/0146491 A1
(43) Pub. Date: May 16, 2019

(54) IN-VEHICLE SYSTEM TO COMMUNICATE WITH PASSENGERS

(71) Applicant: GM GLOBAL TECHNOLOGY OPERATIONS LLC, Detroit, MI (US)

(72) Inventors: Yao Hu, Sterling Heights, MI (US); Xinyu Du, Oakland Township, MI (US); Azeem Sarwar, Rochester Hills, MI (US)

(21) Appl. No.: 15/809,058

(22) Filed: Nov. 10, 2017

(51) Int. Cl. G05D 1/00; G07C 5/08; B60W 40/08; B60W 50/08; G06F 3/01; G06F 3/16; A61B 5/00

(52) U.S. Cl. CPC G05D 1/0088; G07C 5/0808; B60W 40/08; B60W 50/082; G06F 3/017; B60W 2540/28; A61B 5/6893; B60W 2040/0872; B60W 2550/12; B60W 2540/02; G06F 3/167

(57) ABSTRACT

A method of processing commands in a vehicle includes receiving a communication from a user. The method further includes determining that the communication is related to health of the vehicle. The method further includes monitoring the vehicle based on the communication. Upon the determination that fault mitigation should be performed, the method further includes arranging maintenance services for the vehicle. The received communication is in a form selected from a voice communication or a gesture-based communication.

### INTRODUCTION

The subject disclosure relates to a method and system for implementing improved communication with passengers of an automotive vehicle.

Most automotive vehicles interact with passengers through the use of physical controls. For example, a vehicle is driven via physical controls (e.g., pedals and a steering wheel). Various systems of a vehicle are controlled via physical controls (e.g., climate control, audio/visual systems, windows, sunroofs, door locks, seat positions, and the like).

As technology improves, there is an increased desire to include additional means of interacting with passengers in a simple and intuitive manner.

### SUMMARY

In one exemplary embodiment, a method of processing commands in a vehicle includes receiving a communication from a user. The method further includes determining that the communication is related to health of the vehicle. The method further includes monitoring the vehicle based on the communication. Upon the determination that fault mitigation should be performed, the method further includes arranging maintenance services for the vehicle. The received communication is in a form selected from a voice communication or a gesture-based communication.

In addition to one or more of the features described herein, further embodiments may include wherein monitoring the vehicle comprises collecting data regarding behavior of the vehicle. Monitoring the vehicle further includes gathering historical data regarding the vehicle. Monitoring the vehicle further includes prompting the user for additional information.

In addition to one or more of the features described herein, further embodiments may include wherein gathering historical data comprises gathering historical data for similar vehicles.

In addition to one or more of the features described herein, further embodiments may include wherein arranging maintenance services for the vehicle comprises programming the vehicle to travel to a maintenance provider.

In addition to one or more of the features described herein, further embodiments may include communicating with the user using a method chosen from voice output and visual output.

In addition to one or more of the features described herein, further embodiments may include wherein determining that the communication is related to health of the vehicle includes receiving voice communication from the user. The method may further include converting the voice communication into machine-readable format. The method may further include using machine-learning algorithms to interpret the voice communication to determine if the voice communication is related to health of the vehicle.

In addition to one or more of the features described herein, further embodiments may include wherein the voice communication utilizes natural language commands.

In one exemplary embodiment, a method of processing commands in a vehicle comprises receiving a communication from a user. The method may further include determining that the communication is related to the user's health. The method may further include monitoring the user's health using communication and/or at least one sensor located in the vehicle based on the communication. Upon the determination that the user should be transported to the emergency medical facility, the method may further include programming the vehicle to drive to the emergency medical facility. The communication is in a form selected from a voice communication or a gesture-based communication.

In addition to one or more of the features described herein, further embodiments may include wherein monitoring the user's health includes determining an identity of the user; collecting profile data regarding the user. Monitoring the user's health may further include asking a series of questions to the user based on the user's communication, the profile data, and the sensor data, using a machine-learning algorithm. The questions are asked via voice commands. Responses to the question are received in a form selected from a voice communication or a gesture-based communication.

In addition to one or more of the features described herein, further embodiments may include determining if the user should be transported to an emergency medical facility. Based on a determination that the user should be transported to the emergency medical facility, further embodiments may include programming the vehicle to drive to the emergency medical facility.

In addition to one or more of the features described herein, further embodiments may include based on a determination that the communication is an involuntary gesture, determining if the involuntary gesture is indicative of a medical condition.

In addition to one or more of the features described herein, further embodiments may include communicating with the user using a method chosen from voice output and visual output.

In addition to one or more of the features described herein, further embodiments may include wherein the voice communication utilizes natural language commands.

In one exemplary embodiment, a method of processing commands in a vehicle comprises: receiving a communication from a user. The method may further include determining that the communication is related to a driving mode of the vehicle. The method may further include setting the driving mode based on the communication. The communication is in a form selected from a voice communication or a gesture-based communication.

In addition to one or more of the features described herein, further embodiments may include wherein setting the driving mode includes determining an identity of the user. Setting the driving mode may further include collecting profile data regarding the user. Setting the driving mode may further include setting the driving mode based on the profile data.

In addition to one or more of the features described herein, further embodiments may include determining weather conditions; and using the weather conditions to set the driving mode.

In addition to one or more of the features described herein, further embodiments may include wherein the voice communication utilizes natural language commands.

The above features and advantages and other features and advantages are readily apparent from the following detailed description when taken in connection with the accompanying drawings.

### BRIEF DESCRIPTION OF THE DRAWINGS

Other features, advantages and details appear, by way of example only, in the following detailed description of embodiments, the detailed description referring to the drawings in which:

FIG. 1 is a block diagram illustrating a system capable of performing one or more embodiments;

FIG. 2 is a flowchart illustrating the operation of one or more embodiments;

FIG. 3 is a flowchart illustrating the operation of one or more embodiments;

FIG. 4 is a flowchart illustrating the operation of one or more embodiments; and

FIG. 5 is a flowchart illustrating the operation of one or more embodiments.

### DETAILED DESCRIPTION

The following description is merely exemplary in nature and is not intended to limit the present disclosure, its application or uses.

In accordance with an exemplary embodiment, one or more embodiments are shown of an in-vehicle system to allow a vehicle to communicate with passengers.

As automotive vehicles become more autonomous, there is less need for physical input from a person to the vehicle. A commonly used scale illustrating levels of autonomous driving includes levels numbered 0 through 5. Level 0 has no driving automation. Level 1 has assistance to the driver. Level 2 has partial driving automation. Level 3 has conditional driving automation. Level 4 has a high-level of driving automation. And level 5 has full driving automation. In general, the higher the level number, the less input is required from a human.

Traditional automotive vehicles utilize physical inputs to direct the operation of the automotive vehicle. These physical inputs include inputs used to drive the car, such as the steering wheel and the pedals. These inputs also include other systems of the vehicle, such as climate control, audio/visual systems, window position, seat position, mirror position, turn signals, transmission controls, and the like. Because the automotive vehicle is under the control of a human, it has become standard to also utilize physical human inputs to control the various systems of the car. This can include dials, levers, knobs, buttons, and the like that are used to operate the systems.

As computer power has increased, there is an increased desire to use voice commands to control devices. The development of autonomous vehicles has increased the computing power of a vehicle and changed the relationship between a human and a vehicle in such a manner that voice control is increasingly more useful.

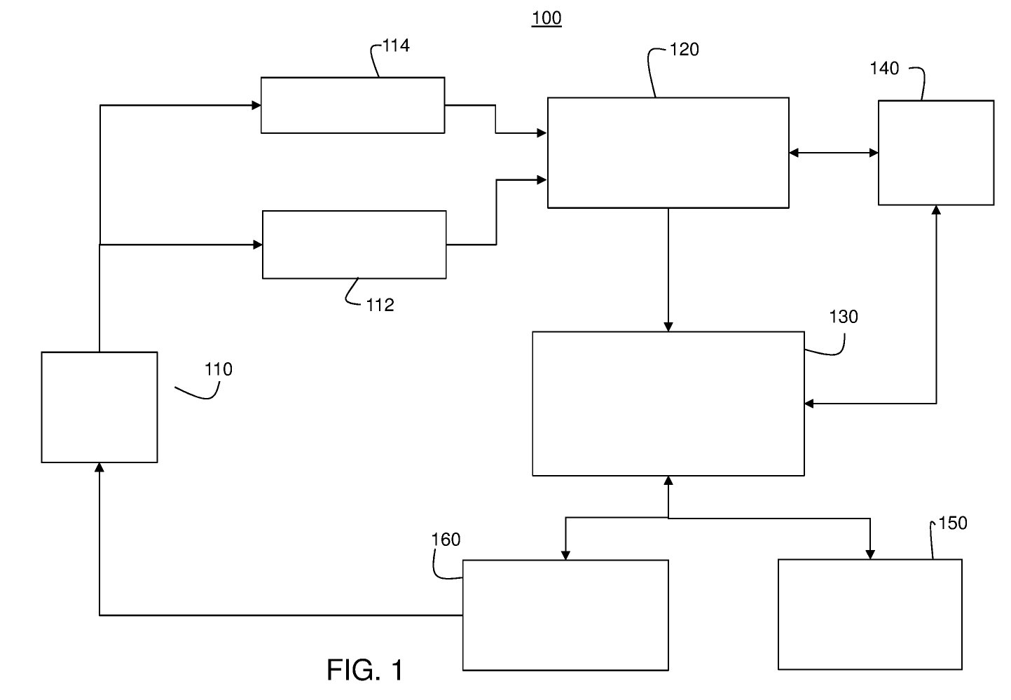

With reference to FIG. 1, a block diagram illustrating an exemplary voice control system 100 of one or more embodiments is presented. Passenger 110 is able to use voice inputs to control various systems of the automotive vehicle in which voice control system 100 is placed. Voice control system 100 includes one or more voice inputs 112. Voice inputs 112 can include one of a variety of different inputs, including audio microphones that are located at various parts of the automotive vehicle. The microphones generate electric signals that represent the received audio. These electric signals can be in the form of digital signals after the electric signals were converted to a digital format for ease of storage and processing. There can also be video inputs 114, such as from a camera, a 3-d sensor or other video sensor, that can provide similar capabilities regarding video inputs.

Communication module 120 receives the electric signals and performs one of a variety of different algorithms on the signals. This can include audio compression, equalization, sound filtering, noise control, and the like. Of particular interest in an automotive vehicle can be noise. Road noise and wind noise is present in some automotive vehicles to a greater extent than exists in a typical home or studio environment. In addition, multiple passengers can result in a need to isolate one voice from other voices. Similar processing can be conducted to video signals.

Communication module 120 also performs speech and gesture recognition functions. Speech recognition allows a system to translate the audio into words that can be used in a variety of different manners. Part of speech recognition can include a voice profile that contains characteristics of a voice that can identify the speaker. In such a manner, a typical passenger of a certain vehicle can have one voice profile while the daughter of the passenger has a different profile. The profile can allow communication modules 120 to more reliably recognize the speech of each user based on characteristics of each user. In addition, communication modules 120 can include machine learning components that allow communication modules 120 to “learn” and adapt to how each user speaks. Recognition and comprehension processor 120 also can include similar capabilities with respect to video signals. For example, gestures can be used by a user and can be slightly different for each user. Thus, the machine learning capabilities of recognition and comprehension processor 120 can be used to more easily distinguish each user and their gestures.

Also included within communication modules 120 are a variety of interfaces with control modules 130 of subsystems that can perform the functions requested by the user. These control modules 130 are embedded computer systems performing one of a variety of different functions, such as engine control, autonomous driving control, vehicle configuration, navigation, diagnostics, telematics, control of vehicle subsystems 150, control of feedback module 160, and the like. The vehicle subsystems 150 can be in one of a variety of different forms, such as actuators, electric motors for mechanical systems (e.g., throttle, brakes, steering, windows, seats, sunroof, doors, locks, and the like). The control modules 130 can also include communication interfaces that allow system 100 to access external computer systems, such as the Internet, or one or more cloud services 140 as well as internal computer systems and storage located throughout the automotive vehicle.

The connection to the Internet and other external computing systems can be accomplished, via a telematics module included in control modules 130, through the use a transceiver coupled to an antenna, wherein the transceiver sends and receives signals using one of a variety of different protocols, such as cellular data protocols (e.g., 4G, LTE, UTMS, WiMAX, and the like), via WiFi, or via global satellite positioning systems (e.g., GPS or GLONASS).

Feedback module 160 includes one or more systems that allow system 100 to communicate with the user. This can include an “Infotainment system,” audio transducers, such as speakers, visual outputs, such as display screens, indicator lights, dials, gauges, and the like. Using feedback module 160, system 100 can indicate statuses to the user, provide updates, and acknowledgments to the user.

Using one or more embodiments including system 100, a variety of different tasks can be initiated by a user through the use of voice commands. These commands can include tasks that control parts of the automotive vehicle that are easily performed, such as “open driver's side window,” “play Mozart Violin Concerto No. 5,” “lower temperature of the car,” “turn on interior lights,” and the like. Once the voice command is understood, the fulfillment of the command is easily performed.

Such commands can include gestures. For example, opening a window might be indicated by a lowering motion of the user's open palm. In some embodiments, the gesture used for each command can be customized by a user. Tasks can include general computing tasks, such as accessing the Internet or performing communication tasks. Exemplary commands can include, “what is on my schedule,” “send message to Sally,” “who won the 1971 World Series,” and various other tasks that can be performed by a smart assistant. Feedback can be provided using speakers, and displays that are part of feedback module 160.

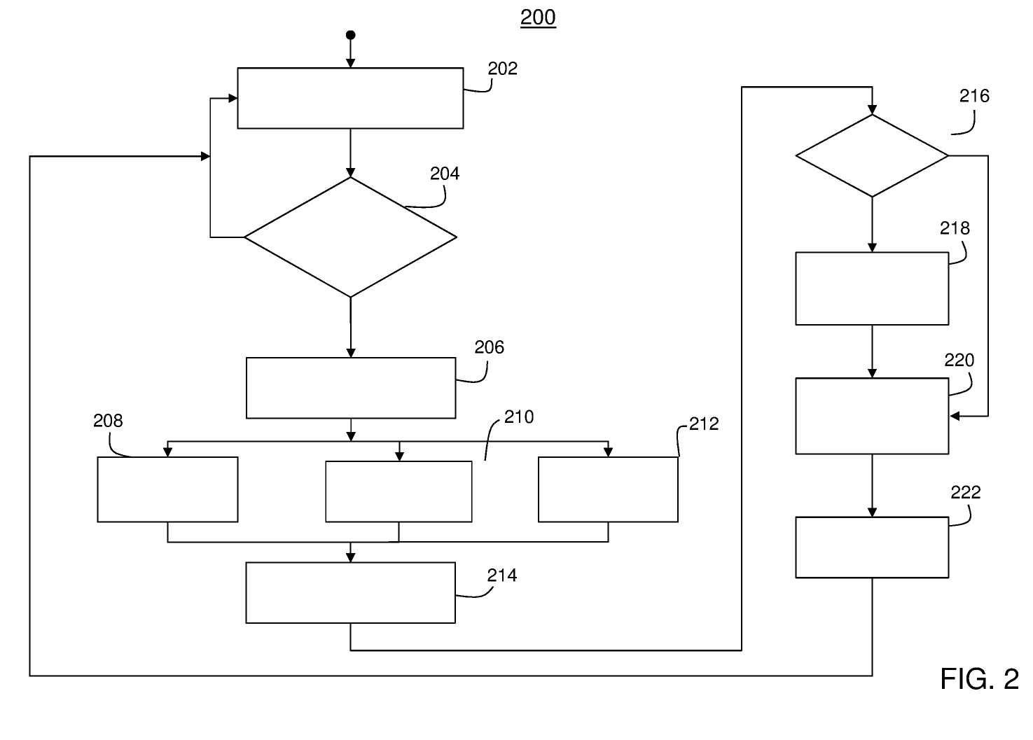

A flowchart illustrating method 200 is presented in FIG. 2. Method 200 is merely exemplary and is not limited to the embodiments presented herein. Method 200 can be employed in many different embodiments or examples not specifically depicted or described herein. In some embodiments, the procedures, processes, and/or activities of method 200 can be performed in the order presented. In other embodiments, one or more of the procedures, processes, and/or activities of method 200 can be combined or skipped. In one or more embodiments, method 200 is performed by a processor as it is executing instructions.

A user's communication is received (block 202). The communication can be via gestures or via voice. The communication is analyzed to determine if the communication is related to the vehicle health (block 204). If not, then method 200 waits until another communication is received.

Once the communication is parsed and recognized (block 206), a variety of actions can occur. Data can be collected to monitor the behavior of the automotive vehicle (block 208). The data can come from a variety of different sources located throughout the automotive vehicle. The sources can include sensors that are configured to collect data of vehicle components behavior. This data can be stored, such as locally or via a cloud service. Historical data can be retrieved, such as locally or via a cloud service (block 210). In some embodiments, the historical data can be restricted to the particular automotive vehicle. In some embodiments, the historical data can include other vehicles, such as for comparison purposes to determine if a subsystem is performing as intended. Additional information can be gathered from the passenger (block 212). The information can be in the form of a series of questions generated using a machine learning algorithm. For example, if the user had reported a sound or vibration coming from a certain location, the user can be asked under what conditions the sound occurs or the exact location of the sound.

The Vehicle Health Management System (VHM) of the automotive vehicle decides on a course of action, based on a variety of criteria (block 214). The information gathered in blocks 208, 210, and 212 can be used to determine the existence, cause, and/or severity of the issue. If an issue exists (block 216), then a course of action can be decided (block 218). Whether there is an issue or not, the system can make a reply to the user (block 220). The reply can be in the form of audio and/or video. For example, a voice indication describing the issue (or lack thereof) can be played through an Infotainment system or via one or more speakers in the automotive vehicle. A visual presentation can be made via a display in the automotive vehicle. Either the video or audio presentation can describe the issue, a suggested course of action, and a request for input. In some cases, the issue might be able to be fixed through a user's actions. In some cases, a visit to a repair facility might be suggested. The location of the nearest repair facility can be relayed to the user, along with available appointment times (retrieved via an Internet connection). The passenger can confirm or acknowledge the report (block 222).

More advanced interactions also are possible. As an example, a user can notice a noise or vibration in the automotive vehicle that did not occur before. The user can say, “I hear a noise coming from the right rear side of the vehicle.” The system will make note of the statement and can collect more data during the noise event. The system can store the statement such that various events can be tracked. The system can make corrections, if possible. The system can contact a repair facility to arrange for a checkup. In an automotive vehicle with advanced autonomous capabilities, the automotive vehicle can even drive to the repair facility depending on the schedule of use of the automotive vehicle.

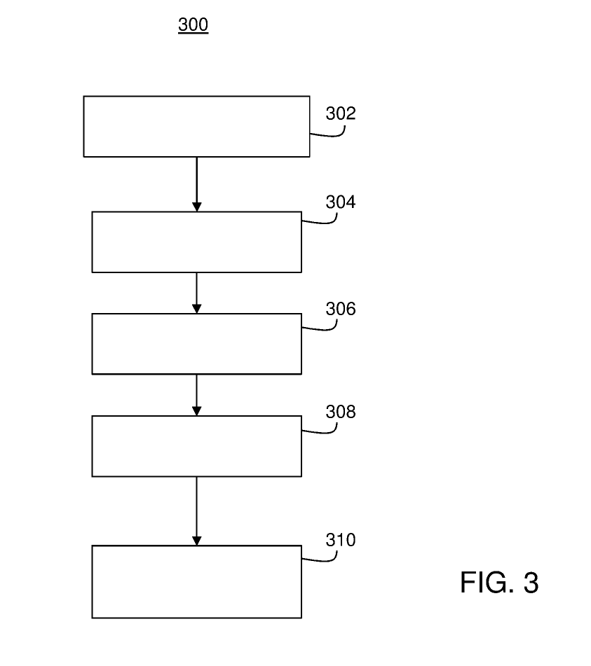

In one or more embodiments, driving controls also can be controlled via voice or gesture commands. A flowchart illustrating method 300 is presented in FIG. 3. Method 300 is merely exemplary and is not limited to the embodiments presented herein. Method 300 can be employed in many different embodiments or examples not specifically depicted or described herein. In some embodiments, the procedures, processes, and/or activities of method 300 can be performed in the order presented. In other embodiments, one or more of the procedures, processes, and/or activities of method 300 can be combined or skipped. In one or more embodiments, method 300 is performed by a processor as it is executing instructions.

For example, a user can set a destination (block 302). The automotive vehicle can determine the vehicle's current location using satellite navigation (block 304) and determine a route to the destination using maps, real-time traffic data, user preferences (e.g., avoid tolls, avoid highways, etc.) and the like (block 306). Once the route is determined, a variety of actions can take place depending on a level of automation of the vehicle. In a vehicle using a high level of automation, the vehicle can commence driving to the destination, with minimal user input (block 308). For lower levels of automation (including no automation at all), directions to the destination can be played to the user via speakers and/or video displays (block 310).

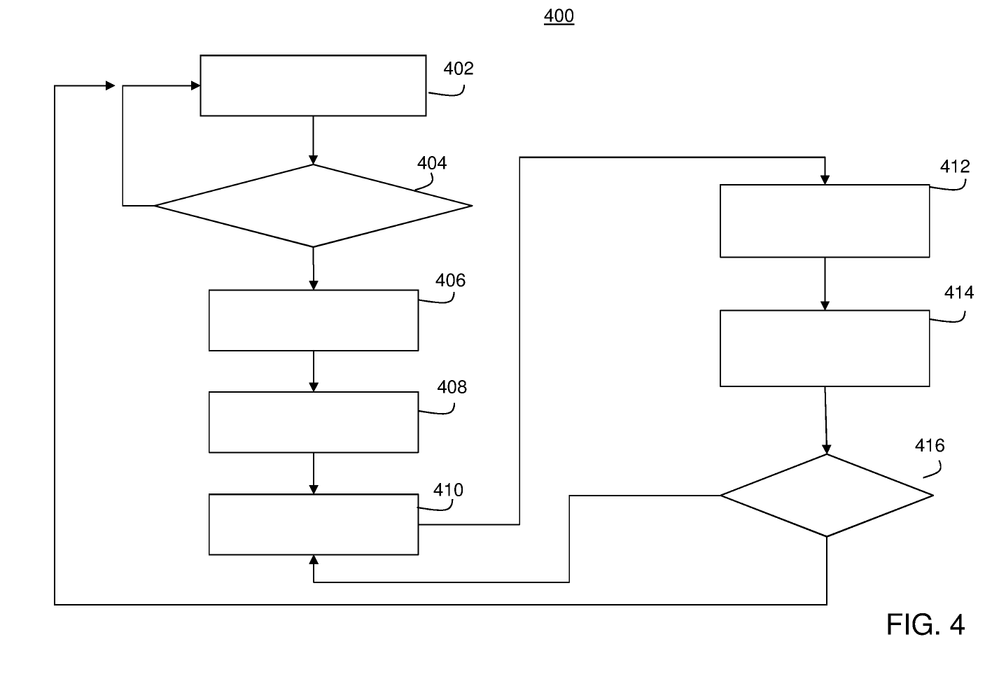

In one or more embodiments, a vehicle can have multiple driving modes. These modes can be switched using voice or gesture commands. A flowchart illustrating method 400 is presented in FIG. 4. Method 400 is merely exemplary and is not limited to the embodiments presented herein. Method 400 can be employed in many different embodiments or examples not specifically depicted or described herein. In some embodiments, the procedures, processes, and/or activities of method 400 can be performed in the order presented. In other embodiments, one or more of the procedures, processes, and/or activities of method 400 can be combined or skipped. In one or more embodiments, method 400 is performed by a processor as it is executing instructions.

Upon receipt of a communication from a user (block 402), it is determined if the passenger is requesting a switch of driving modes (block 404). A vehicle can have multiple driving modes. For example, a vehicle can have a sport mode, with a firmer suspension and less restrictions on the performance of the engine. A vehicle can have an economy mode that contains more restrictions on performance (e.g., avoiding high engine RPMs or fast acceleration). Additional modes can be present, such as a city mode that restricts the top speed of the vehicle. The communication is recognized as a mode change request (block 406).

It should be understood that requests need not be made in a specific, formal language. Machine learning can be used to translate “natural language” to mode change commands. For example, a request to “take it easy” or to make the drive “more relaxing” can be interpreted to be a request to change out of a sport mode.

Modes can be dependent on driving conditions. For example, sensors in the automotive vehicle can determine an outdoor temperature. Sensors can also determine the presence of moisture in the form of rain or snow. Sensors along the drive train can determine if slipping is occurring, possibly due to ice. Based on the driving conditions, some modes can be made available or unavailable to the user. For example, a sport mode might not be allowed below a certain temperature (because of the danger of ice) or in the presence of snow or rain.

Modes can be specific to certain users. For example, one user might not desire to have a sport mode while another user might not desire to use a city mode. The various preferences of the user can be stored locally or via a cloud connection. So a part of the block 406 can be determining which user is present and making the communication. Once the user who made a request is determined, the user's profile can be retrieved (block 408). This retrieval can be from a local storage or from cloud storage. As discussed above, an automotive vehicle might have multiple users. Each user of a car can have a profile. Based on machine learning algorithms, a request for “more pep” can be interpreted to be a request to enter sport mode for one user but be interpreted to be a request to enter a mode short of sport mode for another user.

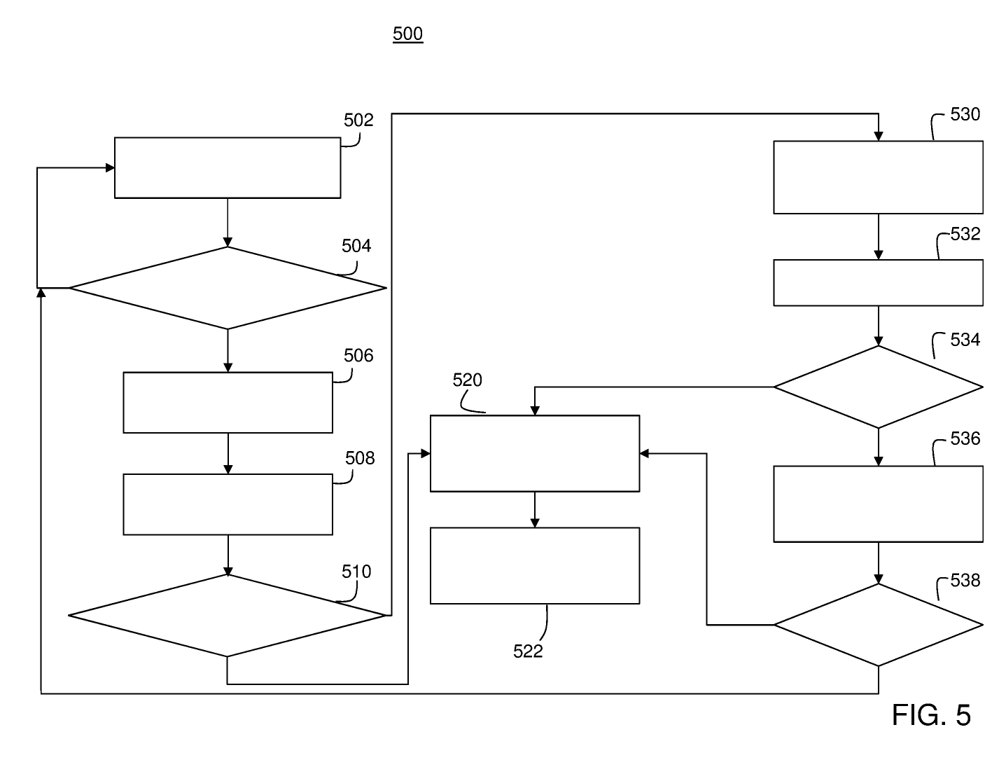

The user's profile can be used to customize driving modes based on the health conditions of the user. A user prone to motion sickness might have a default driving mode being more relaxed than another user who prefers a sport mode. A user who is currently sick (see, e.g., FIG. 5 and the accompanying text) also can have a more relaxed driving mode.

Based on the above information, the desired configuration is determined (block 410). Thereafter, the configuration of the automotive vehicle is changed (block 412). This configuration change can occur in one of a variety of methods now known or those developed in the future. As described above, the configuration change can include a change to suspension characteristics of the automotive vehicle, to the engine of the automotive vehicle, and other subsystems of the automotive vehicle.

The system can make a status report to the user (block 414). The reply can be in the form of audio and/or video. For example, a voice indication describing the new mode can be played through an Infotainment system or via one or more speakers in the automotive vehicle. A visual presentation can be made via a display in the automotive vehicle. The passenger can confirm or acknowledge the report and indicate if he is satisfied with the mode change (block 416). If not, the system can return to block 410. Otherwise, the system can wait for additional input in block 402.

In one or more embodiments, the physical health of a user can be addressed. A flowchart illustrating method 500 is presented in FIG. 5. Method 500 is merely exemplary and is not limited to the embodiments presented herein. Method 500 can be employed in many different embodiments or examples not specifically depicted or described herein. In some embodiments, the procedures, processes, and/or activities of method 500 can be performed in the order presented. In other embodiments, one or more of the procedures, processes, and/or activities of method 500 can be combined or skipped. In one or more embodiments, method 500 is performed by a processor as it is executing instructions.

Upon receipt of a user's communication (block 502), the communication can be examined to determine if the communication is regarding a health concern (block 504). If not, then operation can resume at block 502, where the system waits additional communications from a user. As above, the user's communication can be in the form of audio commands or physical gestures. Audio commands can be processed using one of a variety of different voice recognition algorithms to translate speech to commands (block 506). As discussed above, audio commands can be in a natural language or conversation language. That is, instead of a user utilizing specific commands (e.g., “initiate health protocol”), the user speaks in the same manner he would speak to another person. The voice recognition protocol parses the natural language and determines what the user means for each voice command.

Part of block 506 includes determining which user is making the request. Once the user has been determined, the user's profile can be retrieved (block 508). This can occur from a local storage or from cloud storage. The user's profile can include a variety of information about the user, including health concerns and chronic conditions. In some embodiments, a system can be coupled to one or more sensors. The sensors can include wearable sensors that track vital signs of the user, such as blood pressure, pulse, body temperature, and the like.

If the user's communication is a request to go to a hospital (block 510), an acknowledgment is transmitted via the automotive vehicle's audio and/or video systems (block 520). Thereafter, a route to the nearest appropriate medical facility is calculated (block 522). In the case of an automated vehicle, the route is initiated.

If the user's communication is not a request to go to a hospital, a series of questions can be asked of the user, based on the user's communication (block 530). The questions are generated based on one or more machine learning algorithms and the user's communication. For example, if the user is feeling faint, the user can be asked a series of questions about what he last ate, how long he has been faint or other symptoms he may be experiencing. Sensors can be used to monitor the health of the user. As described above, sensors can include wearable sensors used by the user and can also include sensors located throughout the automotive vehicle. Using the responses to the questionnaires and the sensors, a diagnosis can be determined (block 532). Based on the severity of the diagnosis, it can be determined if the user needs to proceed to an emergency medical facility (block 534). If so, operation can proceed to block 520. Otherwise, a notice is made via the automotive vehicle's audio and/or video systems (block 536). The user can then be asked again if he want to proceed to an emergency medical facility (block 538). If so, operation can resume at block 520. Otherwise, operation can resume at block 502.

In some embodiments, there can be continuous monitoring of the user. For example, if the user had indicated a certain set of symptoms, appropriate sensors can be monitored to determine if the user's conditioning is worsening. Video sensors, such as cameras and three-dimensional sensors, can be monitored to determine if the user needs assistance. For example, a user who experiences sudden movements could be having a seizure. The user's profile could indicate whether or not the user is susceptible to seizures, which would allow a system to more closely monitor such types of movements.

While the above disclosure has been described with reference to exemplary embodiments, it will be understood by those skilled in the art that various changes may be made and equivalents may be substituted for elements thereof without departing from its scope. In addition, many modifications may be made to adapt a particular situation or material to the teachings of the disclosure without departing from the essential scope thereof. Therefore, it is intended that the present disclosure not be limited to the particular embodiments disclosed, but will include all embodiments falling within the scope thereof

What is claimed is:

1. A method of processing commands in a vehicle comprising:
receiving a communication from a user;
determining that the communication is related to health of the vehicle;
monitoring the vehicle based on the communication; and
upon the determination that fault mitigation should be performed, arranging maintenance services for the vehicle, wherein the received communication is in a form selected from a voice communication or a gesture-based communication.

2. The method of claim 1 wherein monitoring the vehicle comprises:
collecting data regarding behavior of the vehicle;
gathering historical data regarding the vehicle; and
prompting the user for additional information.

3. The method of claim 2 wherein gathering historical data comprises gathering historical data for similar vehicles.

4. The method of claim 1 wherein arranging maintenance services for the vehicle comprises programming the vehicle to travel to a maintenance provider.

5. The method of claim 1 further comprising communicating with the user using a method chosen from voice output and visual output.

6. The method of claim 1 wherein determining that the communication is related to health of the vehicle comprises:
receiving voice communication from the user;
converting the voice communication into machine-readable format; and
using machine-learning algorithms to interpret the voice communication to determine if the voice communication is related to health of the vehicle.

7. The method of claim 1 wherein the voice communication utilizes natural language commands.

8. A method of processing commands in a vehicle comprising:
receiving a communication from a user;
determining that the communication is related to the user's health;
monitoring the user's health using communication and/or at least one sensor located in the vehicle based on the communication; and
upon the determination that the user should be transported to emergency medical facility, programming the vehicle to drive to the emergency medical facility, wherein the communication is in a form selected from a voice communication or a gesture-based communication.

9. The method of claim 8 wherein monitoring the user's health comprises:
determining an identity of the user;
collecting profile data regarding the user; and
asking a series of questions to the user based on the user's communication, the profile data, and the sensor data, using a machine-learning algorithm, wherein, the questions are asked via voice commands and responses to the question are received in a form selected from a voice communication or a gesture-based communication.

10. The method of claim 9 further comprising:
determining if the user should be transported to an emergency medical facility; and
based on a determination that the user should be transported to the emergency medical facility, programming the vehicle to drive to the emergency medical facility.

11. The method of claim 10 wherein determining if the user should be transported to an emergency medical facility includes asking the user if the user desires to be transported to the emergency medical facility.

12. The method of claim 8 further comprising based on a determination that the communication is an involuntary gesture, determining if the involuntary gesture is indicative of a medical condition.

13. The method of claim 8 further comprising communicating with the user using a method chosen from voice output and visual output.

14. The method of claim 8 wherein the voice communication utilizes natural language commands.

15. A method of processing commands in a vehicle comprising:
receiving a communication from a user;
determining that the communication is related to a driving mode of the vehicle; and
setting the driving mode based on the communication, wherein the communication is in a form selected from a voice communication or a gesture-based communication.

16. The method of claim 15, wherein setting the driving mode comprises:
determining an identity of the user;
collecting profile data regarding the user; and
setting the driving mode based on the profile data.

17. The method of claim 16 further comprising:
determining weather conditions; and
using the weather conditions to set the driving mode.

18. The method of claim 15 wherein the voice communication utilizes natural language commands.
